(19) United States
(12) Patent Application Publication
(10) Pub. No.: US 2026/0069453 A1
(43) Pub. Date: Mar. 12, 2026

(54) Elevation-Dependent Pneumatic Vitrectomy System

(71) Applicant: Bausch & Lomb Ireland Limtied, Dublin (IE)

(72) Inventors: James T. PERKINS, St. Peters, MO (US); David Robert Jr. FLIEG, Ballwin, MO (US)

(21) Appl. No.: 18/377,426

(22) Filed: Oct. 6, 2023

Related U.S. Application Data
(60) Provisional application No. 63/413,714, filed on Oct. 6, 2022.

(51) Int. Cl. A61F 9/007

(52) U.S. Cl. CPC A61F 9/00736

(57) ABSTRACT

An elevation-dependent surgical pneumatic cutting system is provided. The system includes an absolute pressure sensor for sensing an ambient pressure. The system also includes a pressure drive chamber that receives pressurized air, a supply outlet supplies pressurized air to a pneumatic vitrectomy handpiece and a relief exhaust line exhausts pressurized air. A proportional relief valve is in communication with the relief exhaust line. The pressurized air is exhausted through the proportional relief valve when a supplied air pressure, sensed by a line pressure sensor, exceeds a threshold opening pressure. The threshold opening pressure and an open position are set based on feedback from the absolute pressure sensor indicative of the ambient pressure, whereby the threshold opening pressure of the proportional valve and the open position of the proportional relief valve are variable based on the sensed ambient pressure.

### FIELD

The present disclosure generally relates to elevation-dependent pneumatic vitrectomy systems, and more particularly, to systems and methods for driving pneumatic vitrectomy handpieces at various elevations, including at high elevations.

### BACKGROUND

This section provides background information related to the present disclosure which is not necessarily prior art.

Pneumatic vitrectomy handpieces (e.g., vitrectomy cutters) rely on injecting pressurized air pulses from a drive chamber into the handpiece to deflect a diaphragm or piston. The deflection of the diaphragm or piston drives an inner cutting member across a port in an outer cutting member to cut tissue that is aspirated into the outer cutting member. The inner cutting member is traditionally driven back by a spring (e.g., a helical metal spring) once sufficient pressure has been released from the drive chamber. Dual pneumatic drives (e.g., push-push drives) are also known to drive the inner cutting member in both directions with air pulses and coordinated exhausting of each drive chamber. Regardless of driving mechanism, the drive pulses must be sufficient to drive the inner cutting member forward and/or back in order to cut the tissue, including at high cut rates of several thousand cuts per minute.

### SUMMARY

This section provides a general summary of the disclosure, and is not a comprehensive disclosure of its full scope or all of its features.

Example embodiments of the present disclosure generally relate to elevation-dependent pneumatic vitrectomy systems. In one example embodiment, a surgical pneumatic cutting system includes a pressure sensor for sensing an ambient pressure (e.g., at the location of the system). The system also includes an enclosed pressure chamber having an inlet for receiving pressurized air, a supply outlet for communicating pressurized air to a pneumatic vitrectomy handpiece, and a relief outlet for exhausting pressurized air from the enclosed pressure chamber (e.g., out of the system). A proportional valve is in communication with the relief outlet of the enclosed pressure chamber. The pressurized air is exhausted from the enclosed pressure chamber through the relief outlet (e.g., out of the system) when a pressure within the enclosed pressure chamber exceeds a threshold opening pressure of the proportional valve. The threshold opening pressure is set based on feedback from the pressure sensor indicative of the ambient pressure, whereby the threshold opening pressure of the proportional valve is variable based on an elevation of the surgical pneumatic cutting system.

In another example embodiment, a surgical pneumatic cutting system includes a pressure sensor for sensing an ambient pressure (e.g., at the location of the system). The system also includes a drive chamber having an inlet for receiving pressurized air, a supply outlet for communicating pressurized air to a pneumatic vitrectomy handpiece, and a relief outlet for releasing pressurized air from the drive chamber (e.g., out of the system). A proportional valve is in communication with the relief outlet of the drive chamber, wherein the pressurized air is released from the drive chamber through the relief outlet (e.g., out of the system) when a pressure within the drive chamber exceeds a set pressure of the proportional valve, wherein the set pressure of the proportional valve is adjusted based on feedback from the pressure sensor indicative of the ambient pressure. Additionally, a pneumatic vitrectomy handpiece is in communication with the supply outlet of the drive chamber, the handpiece including an outer cutting member and an inner cutting member positioned within the outer cutting member, wherein the pressurized air drives the inner cutting member.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a block diagram of an example embodiment of an elevation-dependent pneumatic vitrectomy system; and FIG. 2 is a pneumatic circuit diagram of another example embodiment of an elevation-dependent pneumatic vitrectomy system.

Corresponding reference numerals indicate corresponding parts throughout the several views of the drawings.

### DETAILED DESCRIPTION

Example embodiments of the present disclosure generally relate to systems and methods for driving a pneumatic vitrectomy handpiece at different elevations, including at high elevations. Current pneumatic vitrectomy systems rely on a fixed control bleed to exhaust pressurized air from the drive chamber in order to reciprocate a cutting member of the handpiece. In particular, current pneumatic vitrectomy systems may include a relief valve in communication with the drive chamber which exhausts pressurized air from the drive chamber at a fixed threshold opening pressure (e.g., 40 psig). When such systems are used at higher elevations (e.g. above 1000 meters), the vitrectomy systems are unable to perform as effectively at high cut rates due to the decreased pressure and decreased density of air at altitude. To account for this difference, current pneumatic vitrectomy systems include an elevation de-rating of vitrectomy performance, indicating this change in performance. Uniquely, the pneumatic vitrectomy systems of the present disclosure modify the relief valve of the drive chamber such that the threshold opening pressure of the valve is variable based on the elevation. In particular, the pneumatic vitrectomy systems of the present disclosure include a pressure sensor (e.g., an absolute pressure transducer, etc.) to sense the air pressure at the location of the system, including at locations having high elevations. Based on the signal from the pressure sensor, the threshold opening pressure of the relief valve is changed and/or controlled to account for the difference in elevation. This elevation-dependent vitrectomy compressor bleed allows higher compressor output pressure, which enables higher cut rates to be maintained for the vitrectomy function at high elevation. Further, this eliminates the need for elevation de-rating of the pneumatic vitrectomy systems of the present disclosure, as the systems are able to perform effectively at elevation.

Example embodiments will now be described more fully with reference to the accompanying drawings. The description and specific examples included herein are intended for purposes of illustration only and are not intended to limit the scope of the present disclosure.

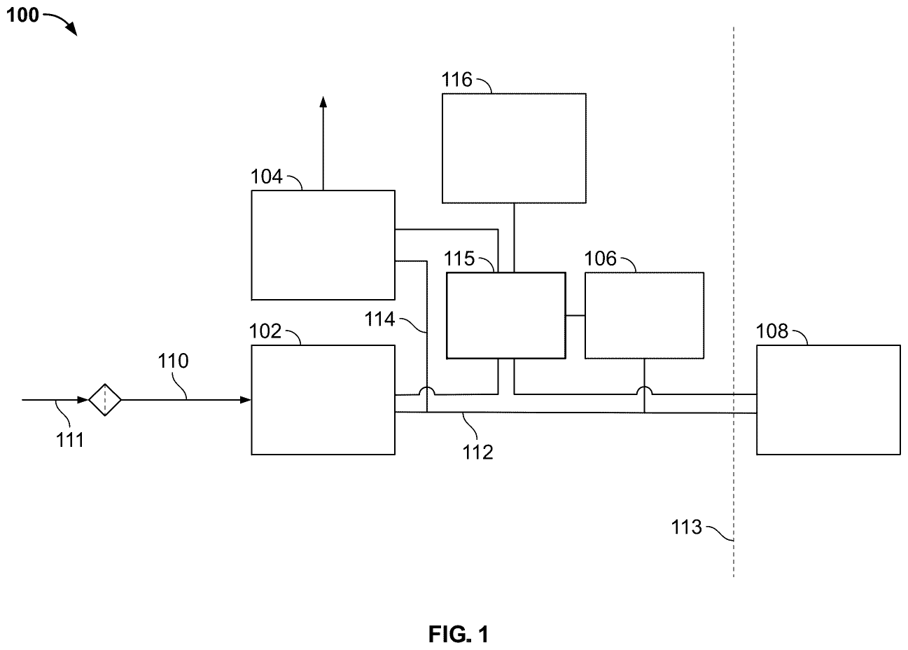

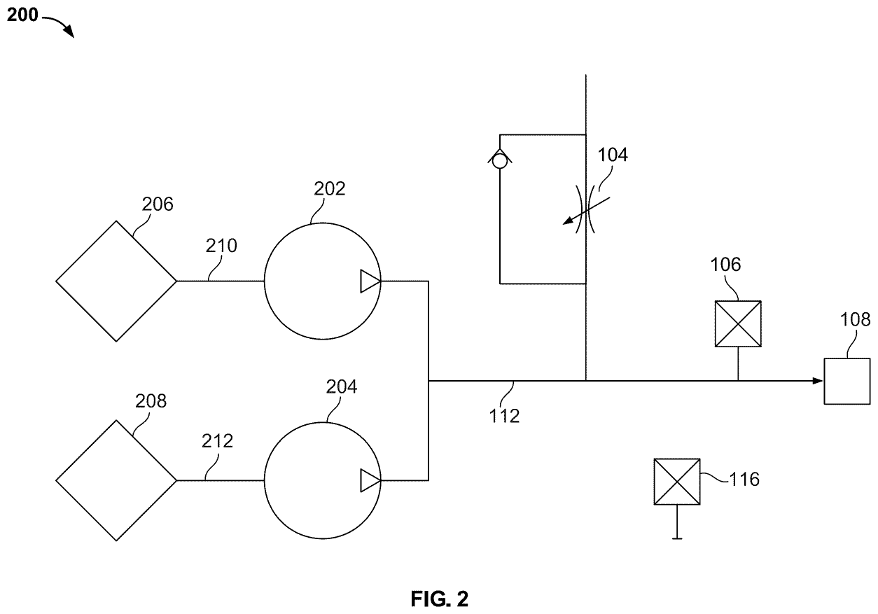

FIGS. 1-2 illustrate example embodiments of surgical pneumatically vitrectomy systems 100, 200 including one or more aspects of the present disclosure. In the illustrated embodiment, the system 100 generally includes a drive chamber 102, a relief/bleed valve 104 in communication with the drive chamber 102, and line pressure sensor 106 for sensing the air pressure in supply line 112. The drive chamber 102 includes an enclosed pressurized chamber that is configured to provide pressurized air pulses to a vitrectomy handpiece 108. The air pulses provided by the drive chamber 102 drive a cutting member of the handpiece 108 (e.g., an inner cutting member, not shown, of the handpiece 108), for example, by deflecting a diaphragm or piston within the handpiece 108. To allow the cutting member of the handpiece 108 to return to its original position (e.g., in a reciprocating manner), pressurized air is released along air supply line 112 through the relief valve 104.

The drive chamber 102 includes an inlet line 110 for receiving pressurized air into the drive chamber 102 through air filter 111. The received pressurized air may be from an on-board air compressor in a surgical console (shown left of dashed line 113) or from an unshown wall-mounted air supply connected to the console 113 via air line 109. The drive chamber may include one or more air compressors and optionally may also include one or more pressure accumulators, as is known. The drive chamber 102 also includes a supply outlet connected to air supply line 112 that, in turn, is in communication with the vitrectomy handpiece 108. To drive the cutting member of the vitrectomy handpiece 108, pressurized air flows into the drive chamber 102 through the inlet line 110 and a pulse of pressurized air flows from the drive chamber 102 through the supply line 112 to the vitrectomy handpiece 108.

The system 100 also includes a relief exhaust line 114 in communication with supply line 112 and the relief valve 104. In particular, pressurized air is exhausted from the supply line 112 through the relief exhaust line 114 when the relief valve 104 is in an open position. The relief valve 104 is selectively opened to maintain the required pressure within the handpiece 108 during operation, including operation at altitude. To maintain the required pressure, the relief valve 104 is opened when a pressure within the supply line 112 exceeds a threshold opening pressure (e.g., a set pressure of the relief valve 104) to allow the cutting member of the vitrectomy handpiece 108 to return/retract in preparation for another pressure pulse from drive chamber 102. The relief valve 104 is a proportional valve allowing for a variety of valve open positions to more finely control the rate of the exhaust air flow, compared to the standard prior art direct solenoid valve that is either open or closed. A control unit is connected to each of the vitrectomy system 100's components for coordinated control of sending pressurized air pulses to handpiece 108 and exhausting air pressure in supply line 112 to allow the cutter member of handpiece 108 to return to its initial open position. Specifically, control unit 115 is in operable communication with the drive chamber 102, proportional relief valve 104, line pressure sensor 106, vit handpiece 108, and absolute pressure sensor 116 for controlling the functioning of vitrectomy handpiece 108. For simplicity and clarity, the aspiration functions and components for the vitrectomy system are not shown or discussed.

The ambient air pressure at the location of the system 100 is measured and adjusted for by use of an absolute pressure sensor 116. An absolute pressure sensor is a sealed device that functions by referencing a perfect or near perfect vacuum, and therefore may produce pressure readings that compensate for the effect of atmospheric pressure, which is especially needed at higher elevations. In particular, the absolute pressure sensor 116 (e.g., a pressure transducer) is in communication with the control unit 115 and may sense a change in elevation based on lower sensed ambient air pressure. Absolute pressure transducer 116 feedback allows the control unit 115 to adjust the proportional valve 104 (controlled bleed) according to the ambient pressure. The control unit 115 can be said to be configured to receive a signal from the absolute pressure sensor indicative of the ambient pressure and to set the threshold opening pressure and the open position of the proportional valve based on the received signal, For example, the higher the elevation the less the proportional valve 103 is opened. When an increase in elevation is sensed and the control unit 115 would compensate for the less dense air by allowing a greater pressure to accumulate in drive chamber 102, eliminating the prior art need for elevation de-rating of vitrectomy performance and enabling high cut rates at higher elevations. The pneumatic design allows for higher compressor output pressures in order to maintain higher cut rates at increased elevation locations. The present disclosure's use of an absolute pressure sensor allows for sensing an ambient pressure level, at the location of the system 100 and, in turn, altering control of the proportional valve and the pressure levels of the drive chamber to be adjusted for elevation changes to maintain high cut rates.

In some embodiments, when the pressurized air is released from the supply line 112 (e.g., when the pressure within the supply line 112 exceeds a threshold pressure, etc.) through the proportional relief valve, a spring (not shown) causes the cutting member (also not shown) of the handpiece 108 to return to its original position. In other embodiments, when the pressurized air is exhausted from the supply line 112 (e.g., when the pressure within the supply line 112 exceeds a threshold opening pressure, etc.), a second drive chamber (not shown) may provide a coordinated pulse of pressurized air to the handpiece 108 to cause the cutting member of the handpiece 108 to return to its original position (e.g., a push-push drive mechanism). In the push-push design each of the drive chambers may have it own supply line to vit handpiece 108 with a separate line pressure sensor and proportional relief valve or there may separate drive chambers but a single supply line to the vit handpiece, depending on design requirements. An example of a two drive chamber, single supply line design is described below with respect to FIG. 2.

In the illustrated embodiment, the valve 104 is a proportional valve. The threshold opening pressure of the valve 104 is variable based on the elevation of the system 100 location. In particular, the threshold opening pressure of the valve 104 is set, and may be adjusted or changed, based on a signal obtained by the absolute pressure sensor 116. The adjustment of pressure levels and valve size openings based on the sensed absolute pressure need only be done once at the beginning of a procedure, however, periodic or continuous adjustments based on the sensed absolute pressure are also acceptable. As described above, the signal obtained by the absolute pressure sensor 116 is indicative of the ambient pressure at the location of the system 100. In this way, the threshold opening pressure of the valve 104 may be varied based on the elevation of the system 100 (e.g., a higher threshold opening pressure may be set at higher elevations, etc.).

Because the absolute pressure sensor 116 senses, detects, measures, etc. the ambient pressure at the location of the system 100, the threshold opening pressure of the valve 104 is optimized for the location, i.e. for the elevation at which the system 100 is being used. When the system is used at high elevation locations, the threshold opening pressure of the valve 104 may be increased to allow a higher compressor output pressure (e.g., to provide an air pulse of a higher pressure to the vitrectomy handpiece 108 through the supply outlet 110) which allows the system 100 to maintain higher cut rates, despite the increased elevation.

FIG. 2 is another example of the present disclosure and is similar to the embodiment of FIG. 1, including use of the same reference numerals where applicable. The components of vitrectomy system 200 have been reduced for clarity and to highlight the main differences. As with FIG. 1 above, no aspiration controls and components are shown or described. In addition, the control unit 115 and its connections are not shown or described with respect to FIG. 2. Those skilled in the art will understand that control of system 200 is similar to control of system 100, except as noted below.

The main difference between system 100 and system 200 is that system 200 makes use of two air compressors 202, 204 as shown. Compressors 202 and 204 are not shown as drive chambers, though they could be part of first and second drive chambers, as in FIG. 1 and are functionally similar. In addition, no accumulators are shown in FIG. 2, although the use of such is acceptable and it is understood that each compressor may be attached to an accumulator. System 200 is particularly useful in driving a push-push type of vitrectomy cutter, as described above. Use of two compressors helps to ensure a pressure pulse of sufficiently high pressure is available to be delivered through supply 112 to drive/push the diaphragm of the vitrectomy handpiece in a direction opposite of the current position of the diaphragm. In between, the drive pulses from compressors 202, 204 the control unit in combination with the line pressure sensor 106 and proportional relief valve 104 will vent alternating sides of the diaphragm to allow the pressure pulses to drive and deflect the diaphragm from the opposite side to cause reciprocating movement of the cutter member of the vit handpiece. As shown, each compressor is in communication with an air inlet line and an air filter 206, 208. As described above, the air inlet lines 210, 212 may receive pressurized air from an on-board air compressor or from a surgical facility's wall air output. The absolute pressure sensor 116 shown in FIG. 2 is connected to the control unit 115 or a similar controller and functions is similar manner as described above.

Elevation-dependent pneumatic vitrectomy systems of the present disclosure allow for effective vitrectomy function (e.g., high cut rates) at increased elevation, by varying the exhaust flow of pressurized air from the supply line based on the elevation (i.e., based on the ambient pressure at the location of the system). In particular, the pneumatic vitrectomy systems of the present disclosure include a relief valve (e.g., a proportional valve) modified by the vitrectomy system, including its threshold opening pressure and flow rate based on a signal from an absolute pressure sensor (e.g., an absolute pressure transducer) that is indicative of the ambient pressure and thus the elevation of the vitrectomy system. Such use of an absolute pressure sensor allows for utilizing a higher threshold pressure at higher elevation to effectively maintain higher cut rates. Doing so eliminates the need for elevation de-rating of vitrectomy performance of pneumatic vitrectomy systems, as was required by prior art vitrectomy systems.

Example embodiments are provided so that this disclosure will be thorough and will fully convey the scope to those who are skilled in the art. Specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” as well as the phrase “at least one of” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper”, “lower” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the present disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the present disclosure, and all such modifications are intended to be included within the scope of the present disclosure.

1. A surgical pneumatic vitrectomy cutting system comprising:
an absolute pressure sensor for sensing an ambient pressure;
a pressure drive chamber having an inlet line for receiving pressurized air, a supply outlet for communicating pressurized air to a pneumatic vitrectomy handpiece through an air supply line;
a relief exhaust line in communication with the air supply line for exhausting pressurized air from the air supply line;
a proportional relief valve in communication with the relief exhaust line;
a line pressure sensor in communication with the air supply line for sensing an air pressure in the air supply line;
wherein the pressurized air is exhausted through the proportional relief valve when an the air pressure, sensed by line pressure sensor in the air supply line exceeds a threshold opening pressure set for the proportional relief valve; and
wherein the threshold opening pressure and a selected variable open position of the proportional relief valve are set based on feedback from the absolute pressure sensor indicative of the ambient pressure, whereby the threshold opening pressure of the proportional valve is variable and the variable open position of the proportional relief valve is variable and selected based on the sensed ambient pressure.

2. The system of claim 1, further comprising a control unit configured to receive a signal from the absolute pressure sensor indicative of the ambient pressure and to set the threshold opening pressure and the open position of the proportional valve based on the received signal.

3. The system of claim 1, further comprising a second pressure drive chamber for driving the pneumatic vitrectomy handpiece in a push-push drive mechanism.

4. The system of claim 1, further comprising the pneumatic vitrectomy handpiece in communication through the air supply line with the pressure drive chamber.

5. The system of claim 4, wherein the pneumatic vitrectomy handpiece includes a cutting member driven from a first position to a second position by pressurized air from the pressure drive chamber and a spring for returning the cutting member from the second position to the first position when the pressurized air is exhausted from the air supply line.

6. A surgical pneumatic cutting system comprising:
an absolute pressure sensor for sensing an ambient pressure;
a drive chamber having an inlet line for receiving pressurized air, a supply outlet connected to an air supply line for communicating pressurized air to a pneumatic vitrectomy handpiece;
a relief exhaust line in communication with the air supply line for exhausting pressurized air from the air supply line;
a proportional relief valve in communication with the relief exhaust line;
a line pressure sensor in communication with the air supply line for sensing an air pressure in the air supply line;
wherein the pressurized air is exhausted through the proportional relief valve when the air pressure, sensed by line pressure sensor, exceeds a threshold opening pressure set for the proportional relief valve;
wherein the threshold opening pressure and an open position are set based on feedback from the absolute pressure sensor indicative of the ambient pressure, whereby the threshold opening pressure of the proportional valve is variable and the open position of the proportional relief valve is also variable based on the sensed ambient pressure; and
a pneumatic vitrectomy handpiece in communication with the air supply line, the handpiece including an outer cutting member and an inner cutting member positioned within the outer cutting member, wherein the pressurized air drives the inner cutting member.

7. The system of claim 6, further comprising a controller configured to receive a signal from the absolute pressure sensor indicative of the ambient pressure and to set the threshold opening pressure and the open position of the proportional relief valve based on the received signal.

8. The system of claim 6, further comprising a second drive chamber for driving the pneumatic vitrectomy handpiece in a push-push drive mechanism.

9. The system of claim 6, wherein the pressurized air drives the inner cutting member from a first position to a second position; and
wherein the pneumatic vitrectomy handpiece includes a spring for returning the inner cutting member from the second position to the first position when the pressurized air is exhausted from the air supply line.

10. A surgical pneumatic cutting system comprising:
an absolute pressure sensor for sensing an ambient pressure;
a drive chamber having an inlet line for receiving pressurized air and an outlet line connected to an air supply line for communicating pressurized air to a pneumatic vitrectomy handpiece;
a relief exhaust line in communication with the air supply line for exhausting pressurized air from the air supply line;
a proportional relief valve in communication with the relief exhaust line, wherein the proportional relief valve has selectable valve size openings;
a line pressure sensor in communication with the air supply line for sensing an air pressure in the air supply line;
wherein the pressurized air is exhausted through the proportional relief valve when the air pressure in the air supply line exceeds a threshold opening pressure set for the proportional relief valve;
wherein the threshold opening pressure and an open position of the selectable valve size openings are set based on feedback from the absolute pressure sensor based on the sensed ambient pressure;
wherein both of (i) the threshold opening pressure and (ii) the open position of the proportional relief valve are variable based on the sensed ambient pressure.

11. system of claim 10, further comprising:
a controller configured to receive a signal from the absolute pressure sensor indicative of the ambient pressure and to set the threshold opening pressure and the open position of the proportional relief valve based on the received signal.

12. The system of claim 11, wherein the controller is configured to continuously receive the signal and set the threshold opening pressure and the open position of the proportional relief valve.

13. The system of claim 11, wherein the controller is configured to control a flow rate of the received pressurized air to the drive chamber.

14. The system of claim 11, wherein the proportional relief valve is a proportional valve allowing for a plurality of the valve open positions;
wherein the plurality of valve open positions are operable to be selected to control a rate of an exhaust air flow as the exhausting pressurized air.

15. The system of claim 11, further comprising:
a pneumatic vitrectomy handpiece in communication with the air supply line, the handpiece including an outer cutting member and an inner cutting member positioned within the outer cutting member;
wherein the pressurized air drives the inner cutting member.
